(19) United States
(12) Patent Application Publication
(10) Pub. No.: US 2011/0281362 A1
(43) Pub. Date: Nov. 17, 2011

(54) Electrotransformation of Gram-Positive, Anaerobic, Thermophilic Bacteria

(75) Inventor: Daniel G. Olson, Norwich, VT (US)

(73) Assignee: Mascoma Corporation, Lebanon,, NH (US)

(21) Appl. No.: 13/122,509

(22) PCT Filed: Oct. 13, 2009
(86) PCT No.: PCT/US09/60501

§ 371 Date: Aug. 2, 2011

Related U.S. Application Data
(60) Provisional application No. 61/109,642, filed on Oct. 30, 2008.

(51) Int. Cl. C12N 15/74; C12N 1/21

(52) U.S. Cl. 435/471; 435/252.3

(57) ABSTRACT

The present invention relates to methods for transforming Gram-positive, anaerobic, thermophilic bacteria via electroporation and Gram-positive, anaerobic, thermophilic bacteria transformed by the disclosed methods. The methods employ voltage pulsing schemes that decrease arcing such that increased transformation efficiency and cell viability is observed. The present invention is further directed to a method for transforming Gram-positive, anaerobic, thermophilic bacteria via electroporation using recovery/selection temperatures to effect increased transformation efficiency in difficult to transform bacteria.

### BACKGROUND OF THE INVENTION

1. Field of the Invention

The present invention relates to methods for genetically engineering microorganisms. In particular, the present invention relates to electrotransformation of Gram-positive, anaerobic, thermophilic bacteria by driving DNA segments across a bacterial cell membrane with an electric field.

2. Background Art

Bacteria typically have genomes including one or a few chromosomal strands of Deoxyribose Nucleic Acid (DNA) having thousands of segments known as genes. Each gene includes a sequence of nucleotides that code for one or more peptides, or proteins, together with regulatory nucleotide sequences such as promoters, start codons, stop codons, and other transcriptional control sequences. Bacteria can also incorporate shorter DNA segments such as plasmids and dormant bacteriophages. These plasmids and bacteriophage can also contain genes and can either reside in the cytoplasm alone or be incorporated into the bacterial genome.

Typical bacterial species encode proteins that have evolved according to the needs of the species in the environment that they normally inhabit. These proteins include proteins for reproduction of the bacterial cell, for energy production, for producing fundamental building blocks of the cell like nucleotides, for producing motility structures like flagella, and for toxins that give that species a competitive advantage over other species in the same environment.

Bacteria have been artificially engineered to produce proteins unnecessary for survival of that bacterial species, but of interest to humans. This has been done by isolating or creating a new segment of DNA encoding a desired protein and inserting the new segment either into the bacterial genome or allowing the new segment to remain and replicate in the bacteria's cytoplasm. Once that segment of DNA is inserted into the bacterial genome, the desired protein can be expressed after appropriate transcription and translation events have taken place. The process of inserting the new DNA segment is known as transformation.

Similarly, transformation can be used to disable selected genes normally present in the bacterial genome, or to increase production of preferred endogenous products. Alteration of bacterial genomes can be of use in adapting a microorganism for survival in a new or different environment, or to modify the microorganism's metabolic pathways to produce non-protein metabolic products of interest to humans.

### Difficulties in Bacterial Cell Transformation

Some bacteria with complex intracellular morphology and/or complex cell development cycles are difficult to electrotransform. When transforming these bacteria, it is important to identify the right growth medium, specific growth stage of the culture, and some other biological conditions, such that the cells become as “electrocompetent” as possible before performing electroporation.

When some bacterial species are subjected to stressful conditions they may form hardy endospores. Endospores are generally smaller than normal vegetative cells, and much more resistant to heat, dessication, radiation, acids, chemical disinfectants, and other environmental hazards than normal vegetative cells. Electroporation may provide sufficient chemical, electrical, and thermal stress to trigger spore formation in some bacteria. As spores form, much of the cellular contents, often including the newly inserted and desired DNA plasmid if sporulation occurs immediately after electroporation, is excluded from the spore. Spore forming bacteria with complex lifecycles therefore are often difficult to transform.

Several genera of endospore-forming bacteria have been classified on the basis of morphology, relationship to oxygen and energy metabolism. The two most frequently discussed genera of endospore-forming bacteria include Bacillus and Clostridium, both of which contain Gram-positive genus members. While Bacillus bacteria are aerobic or facultatively aerobic, Clostridium bacteria are strictly anaerobic. Clostridium includes several species that are difficult to transform and typically derive energy through fermentation and for which free oxygen is toxic. Example members of the genus include C. perfringens, C. botulinum, as well as C. thermocellum, which is capable of fermenting cellulose at a temperature of 60° C. Other members of the genus include C. cellobioparum, which is also capable of fermenting cellulose; C. butyricum, C. acetobutylicum, C. pasteurianum, and C. thermosulfurogens, which are capable of fermenting sugars, starch and pectin; C. aceticum, C. formicoaceticum, C. formicoaceticum, and C. methylpentosum, which are capable of fermenting pentose and methylpentose molecules; C. sporogenes, C. tetani, C. tentanomorphum, and C. propionicum, which are capable of fermenting proteins and amino acids; C. bifermentans, which is capable of fermenting carbohydrates or amino acids; C. acidurici, which is capable of fermenting purine molecules; and C. kluyveri, which is capable of fermenting ethanol to produce fatty acids. (See T. D. Brock, Biology of Microorganisms, 7^{th} ed., Prentice Hall, Englewood Cliffs, p. 801.)

Examples of Gram-positive, anaerobic thermophiles that have been successfully transformed are limited to Thermoanaerobacterium species and a few reports of C. thermocellum and C. thermosaccharolyticum transformation. In these studies, the highest efficiencies of electrotransformation were obtained by Mai (˜10³ CFU/μg DNA) for Thermoanaerobacterium sp. strain JW/SL-YS485, Tyurin (2.8×10⁵ CFU/μg DNA) for C. thermocellum strain DSM 1313, and Tyurin (˜7.42×10⁵ CFU/μg DNA) for Thermoanaerobacterium saccharolyticum strain YS485. All of the protocols of these studies featured incubation in the presence of either isoniacin or derivatives thereof. (See Mai V. et al., FEMS Micrbiology Letters 148:163-167 (1997); Tyrurin, M. V. et al., Appl. Environ. Microb. 70:883-890 (2004); and Tyrurin M. V. et al., Appl. Environ. Microb. 71:8069-8076 (2005).)

Prior techniques for transformation of difficult-to-transform bacteria have included modifying the bacterial cell walls by growing the bacteria in media containing ingredients that damage developing cell walls, by partially digesting the cell walls and/or using elaborate voltage pulsing schemes and apparati to transform these bacteria. Ingredients that have been used in the past to weaken the cell wall and enhance electrotransformation efficiency include glycine, muralytic enzymes and/or isonicotinic acid hydrazide (isoniacin). In situations where such ingredients have been used, the weakened cell walls allow desired DNA plasmids to reach the cell membrane more rapidly through the electopores created during electroporation. It has been found that weakening cell walls often adversely affects viability of the bacteria to the extent that electroporation yield or transformation efficiency remains unacceptably low.

It is desirable to improve transformation yield of difficult-to-transform bacteria to expedite research performed with such bacteria.

Genetically Modified C. thermocellum in Biofuel Production

Mammals, yeast, and other eukaryotic organisms lack enzymes for hydrolyzing cellulose; sugars linked with beta-glucoside bonds in cellulose are not metabolized and often become waste. Grass, wood, agricultural residues (including cornstalks, wheat and oat straw, and manure), and municipal solid waste (paper), for example, have high cellulose content.

C. thermocellum has the ability to hydrolyze cellulose; it ferments the resulting sugars into a mixture of acetone, alcohols and organic acids, including acetic acid (reviewed in E.P. Cato, Bergey's Manual of Systematic Bacteriology, 2^{nd} ed., vol. 2. Williams & Wilkins, Baltimore). C. thermocellum also exhibits one of the highest described growth rates on cellulose.

While the preferred commercial method of acetone and butanol production currently is via chemical synthesis using petroleum products, there is considerable industrial interest in the production of ethanol (an automotive fuel additive) by the bacterial fermentation of cellulose. Genetic studies of Clostridium are underway to increase the yield of ethanol and reduce the formation of acidic fermentation products, so that the goal of converting waste cellulose to useful motor fuel can be realized on a commercial scale.

Although the genome of C. thermocellum has been completely sequenced and a number of C. thermocellum genes have been cloned into other bacteria, reliable methods have not been established for the introduction of foreign genes into this microorganism. The absence of such methods has been a significant impediment to studies of C. thermocellum aimed at increasing both fundamental understanding and applied capability, especially as multiple, substantial, genome modifications are required to render C. thermocellum suitable for use in the industrial production of ethanol from cellulose.

Therefore, there is a need for improved techniques for transforming Gram-positive, anaerobic, thermophilic bacteria via electroporation. Methods that can be followed without the use of cell-weakening agents are desirable.

### BRIEF SUMMARY OF THE INVENTION

In one aspect, the present invention is directed to a method for transforming DNA into Gram-positive, anaerobic, thermophilic bacteria by electroporation which includes preparing a suspension of bacteria with DNA and applying a voltage burst between a first electrode and a second electrode such that an electric field is applied to the bacterial/DNA suspension such that transformation occurs, wherein the voltage burst comprises one or more identical square pulses that have a duration from about 10 μs to about 3 ms.

Another aspect of the present invention is directed to a method for transforming DNA into Gram-positive, anaerobic, thermophilic bacteria by electroporation which includes preparing a suspension of bacteria with DNA and applying a voltage burst between a first electrode and a second electrode such that an electric field is applied to the bacteria/DNA suspension such that transformation occurs, wherein the resulting transformed bacteria are cultured at a recovery temperature followed by culture at a selection temperature, and wherein the recovery temperature is lower than the selection temperature.

In some embodiments of the present invention the voltage burst is one square pulse that has a duration from about 10 μs to about 3 ms. In other embodiments, the voltage burst comprises from about 30 to about 100 identical square pulses having a duration from about 10 μs to about 3 ms. In another embodiment, the voltage burst comprises from about 30 to about 50 identical square pulses having a duration from about 10 μs to about 3 ms.

In some embodiments of the present invention the voltage burst is a square pulse with a field strength from about 10 kV/cm to about 20 kV/cm. In other embodiments, the square pulse or identical square pulses have field strength from about 15 kV/cm to about 30 kV/cm. In other embodiments, the voltage burst has a duration from about 30 μs to about 1.5 ms.

In some embodiments of the present invention, the Gram-positive, anaerobic, thermophilic bacteria to be transformed are cultured prior to electroporation to a cell density OD600 of from about 0.3 to about 1.0. The bacteria can be cultured prior to electroporation from about 12 to about 18 hours.

In some embodiments of the present invention, the Gram-positive, anaerobic, thermophilic bacteria are cultured for at least about 12 hours after electroporation at a recovery temperature. In other embodiments, the bacteria can be cultured for at least about 22 hours after electroporation at a recovery temperature.

In some embodiments of the present invention, the Gram-positive, anaerobic, thermophilic bacteria are cultured after electroporation at a recovery temperature of from about 25° C. to about 52° C. In other embodiments, the bacteria are cultured after electroporation at a recovery temperature of from about 40° C. to about 52° C. In yet another embodiment, the bacteria are cultured after electroporation at a recovery temperature of about 51° C.

In some embodiments of the present invention, the Gram-positive, anaerobic, thermophilic bacteria are cultured after electroporation and recovery at a selection temperature of from about 52° C. to about 64° C. In other embodiments, the bacteria are cultured at a selection temperature of about 55° C.

In some embodiments of the present invention, the Gram-positive, anaerobic, thermophilic bacteria are cultured at a selection temperature from about 36 to about 72 hours. In other embodiments, the bacteria are cultured from about 48 to about 72 hours.

In an additional embodiment of the present invention, the electroporation method further comprises selecting transformed Gram-positive, anaerobic, thermophilic bacteria by incubating the transformed bacteria on media containing at least one antibiotic for which a transformed bacterium is resistant and for which a non-transformed bacterium is susceptible. In another embodiment, the media contains thiamphenicol.

In some embodiments of the present invention, the Gram-positive, anaerobic, thermophilic bacteria are not treated with isoniacin prior to electroporation.

In one embodiment of the present invention, the Gram-positive, anaerobic, thermophilic bacteria are endospore-forming. In another embodiment, the bacteria are selected from the genus Clostridium or Thermoanaerobacterium. In yet another embodiment, the bacteria is a Clostridium thermocellum strain. In another embodiment, the bacteria is a Clostridium thermocellum strain selected from the group consisting of DSM 1313, DSM 1237 and DSM 2360.

In some embodiments of the present invention, the transforming DNA is contained within an expression vector. In other embodiments, the expression vector is a plasmid. In other embodiments, the expression vector comprises a DNA sequence of an endogenous gene of the Gram-positive, anaerobic, thermophilic bacterium to be transformed. In other embodiments, the expression vector comprises a DNA sequence of an exogenous gene to the Gram-positive, anaerobic, thermophilic bacterium to be transformed. In additional embodiments, the expression vector comprises a chloramphenicol acetyltransferase gene.

In some embodiments of the present invention, the Gram-positive, anaerobic, thermophilic bacteria are transformed with a transformation efficiency of from about 8×10³ CFU/μg DNA (colony forming units per μg of DNA transformed) to about 5×10⁵ CFU/μg DNA.

Another embodiment of the present invention is directed to a Gram-positive, anaerobic, thermophilic bacterium transformed with an expression vector by the claimed transformation methods.

Further embodiments, features, and advantages of the present inventions, as well as the operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate one or more embodiments of the present invention and, together with the description, further serve to explain the principles of the invention and to enable a person skilled in the pertinent art to make and use the invention.

FIG. 1 shows maps of plasmids (A) pNW33N and (B) pMU102 used in electrotransforming Gram-positive, anaerobic, thermophilic bacteria. The pNW33N plasmid contains an origin of replication, (Rep Origin 1), a catalase gene (cat) and two repeat regions (identified as Repeat Region A and Repeat Region B on the plasmid map). The repeats share 100% homology with one another over 358 by and are in opposite DNA orientation. Repeat Region B is flanked by Fok I and Eco RI endonuclease restriction sites. To generate pMU102, pNW33N was digested with Fok I and Eco RI to remove a 653 by fragment that included Repeat Region B. The digested pNW33N plasmid was then Klenow-treated and religated. The newly-generated pMU102 plasmid was then transformed into E. coli.

FIG. 2 shows the effect of pulse duration on transformation efficiency. C. thermocellum DSM 1313 cells were prepared for electrotransformation as described in Example 1. The pulse setup parameters were varied so that cells were electroporated with 333 3 μs identical pulses with a field strength of 30 kV/cm, 100 identical pulses with a duration of 10 μs and a field strength of 22.5 kV/cm, 33 identical pulses with a duration of 30 μs and a field strength of 22.5 kV/cm, 11 identical pulses with a duration of 90 μs and a field strength of 20 kV/cm, 3 identical pulses with a duration of 333 μs and a field strength of 20 kV/cm, or a 1 ms pulse with a field strength of 17.5 kV/cm. FIG. 2A lists the number of colonies observed after 2-3 days of incubation on selection media and the transformation efficiency of each pulsing scheme is reported in the number of colonies of transformants observed per μg DNA. FIG. 2B shows that pulsing scheme 3 produced the most number of transformants with a transformation efficiency of 1.31×10⁴ CFU/μg DNA.

FIG. 3 shows transformation efficiencies of plasmid DNA extracted from C. thermocellum and E. coli. DNA of plasmids pMU102 and pNW33N was extracted from C. thermocellum and E. coli cells that had previously been transformed with pMU102 or pNW33N. Untransformed C. thermocellum DSM 1313 cells were prepared for electroporation as described in Example 1 with DNA of plasmid pMU102 or pNW33N, extracted from either C. thermocellum or E. coli cells. Higher transformation efficiencies were observed when DNA extracted from C. thermocellum was used for electrotransformation.

FIG. 4 shows the transformation efficiencies of C. thermocellum M0074 cells with pMU102 plasmid DNA. C. thermocellum M0074 cells were electrotransformed according to the methods described in Example 2. A transformation efficiency of 2.5 CFU/μg DNA was observed when cells were electroporated with pMU102 plasmid DNA extracted from E. coli.

FIG. 5 shows the transformation efficiencies of C. thermocellum M0042 cells with pMU102 plasmid DNA. C. thermocellum M0042 cells were electrotransformed according to the methods described in Example 3. A transformation efficiency of 2.5 CFU/μg DNA was observed when cells were electroporated with one square pulse having a duration of 1.5 ms and a field strength of 15 kV/cm.

FIG. 6 shows the transformation efficiencies of C. thermocellum M0043 cells with pMU102 plasmid DNA. C. thermocellum M0043 cells were electrotransformed according to the methods described in Example 4. A transformation efficiency of 2.5.×10⁴ CFU/μg DNA was observed when cells were electroporated with one square pulse having a duration of 1.5 ms and a field strength of 15 kV/cm.

FIG. 7 shows the comparable transformation efficiencies observed when C. thermocellum DSM 1313 cells were electrotransformed in custom-built and commercially-available electroporators. The cells were electrotransformed according to the methods described in Example 5. A transformation efficiency of 1.3×10⁴ CFU/μg DNA was observed when cells were electroporated in a custom high voltage capacitor-insulated gate bipolar transistor (IGBT) switch electroporator. A transformation efficiency of 1.0×10⁴ CFU/μg DNA was observed when cells were electroporated in a BioRad Gene Pulser Xcell electroporator.

FIG. 8 shows the comparable transformation efficiencies observed when C. thermocellum M0003 cells were electrotransformed in custom-built and commercially-available electroporators. The cells were electrotransformed according to the methods described in Example 6. A transformation efficiency of 8.1×10³±6.5×10³ CFU/μg DNA was observed when cells were electroporated in a custom high voltage capacitor-IGBT switch electroporator. A transformation efficiency of 1.6×10⁴±8.9×10³ CFU/μg DNA was observed when cells were electroporated in a BioRad Gene Pulser Xcell electroporator.

FIG. 9 shows the transformation efficiencies observed when C. thermocellum M0003 cells were electrotransformed in a custom high voltage-capacitor-IGBT switch electroporator with various wash buffers. The cells were electrotransformed according to the methods described in Example 7. A transformation efficiency of 4.9×10⁵ CFU/μg DNA was observed when cells had been washed with pure water. A transformation efficiency of 2.6×10⁵ CFU/μg DNA when cells had been washed with 50 mM xylose solution. A transformation efficiency of 2.5×10⁵ CFU/μg DNA when cells had been washed with 50 mM xylose, resazurin solution. A transformation efficiency of 1.8×10⁴ CFU/μg DNA when cells had been washed with 50 mM xylose, resazurin, cysteine solution. A transformation efficiency of 5.1×10³ CFU/μg DNA when cells had been washed with 500 mM sucrose solution. A transformation efficiency of 2.1×10⁵ CFU/μg DNA when cells had been washed with 50 mM autoclaved xylose solution.

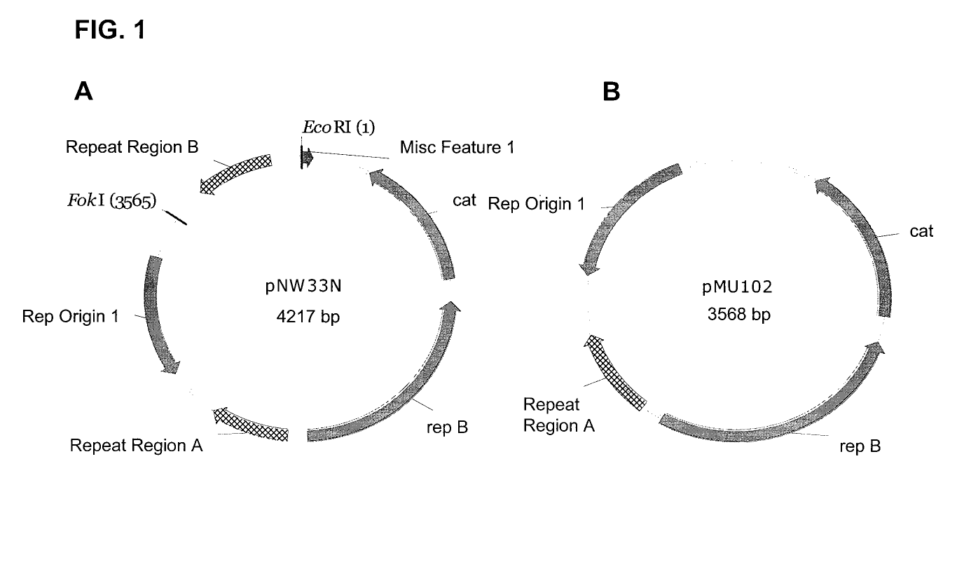

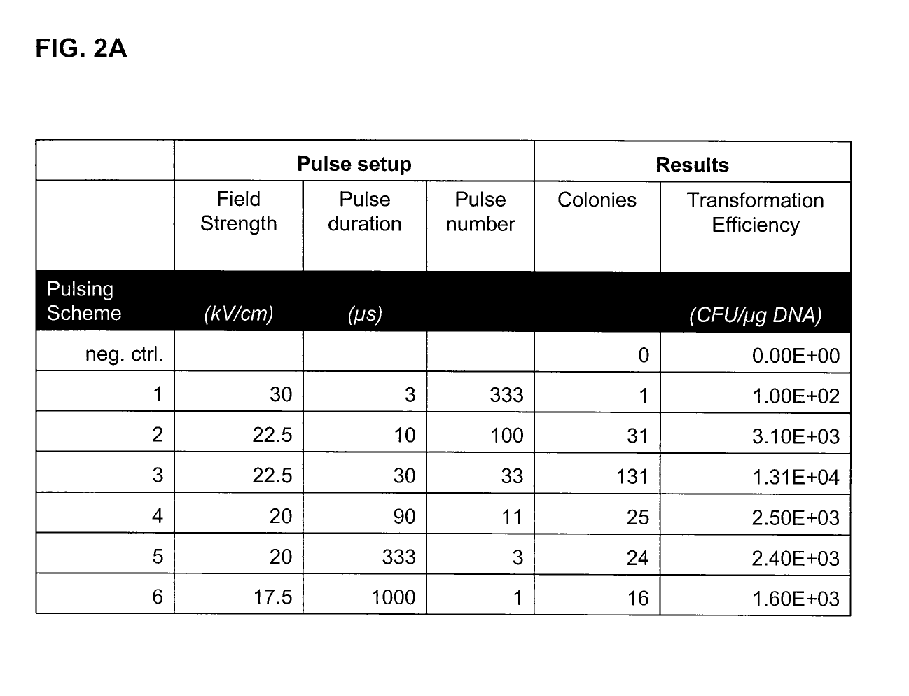

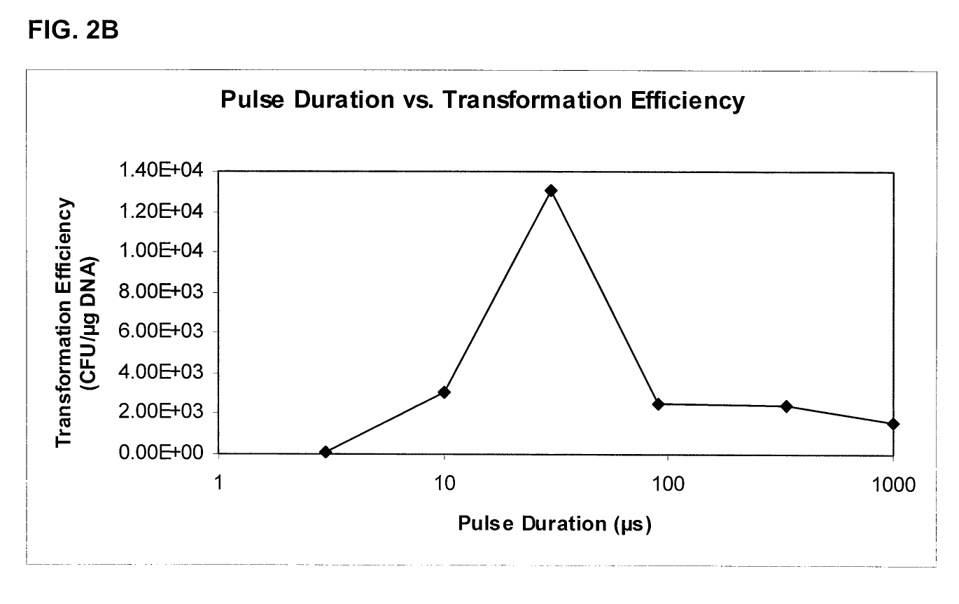

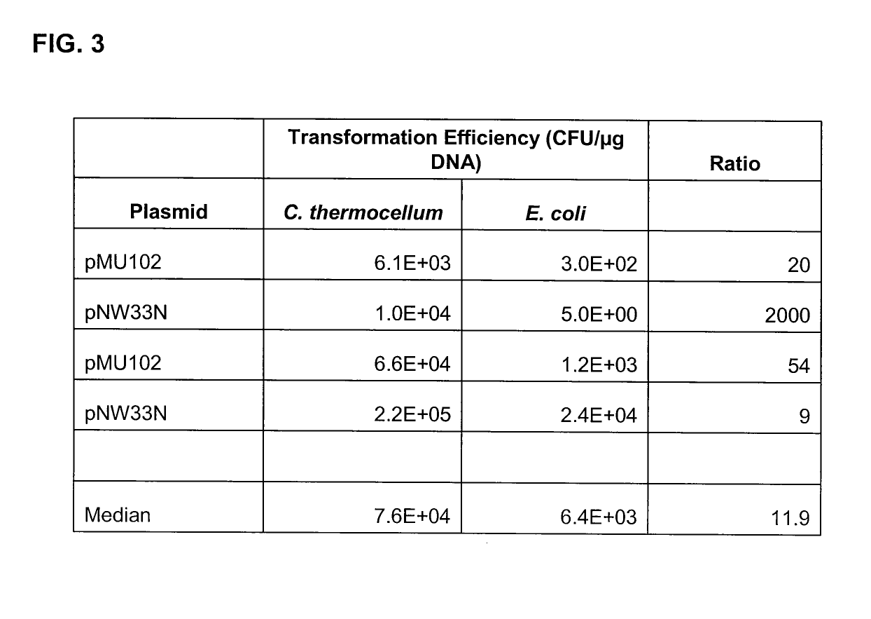

### DETAILED DESCRIPTION OF THE INVENTION

The present invention provides methods that are useful for genetically engineering Gram-positive, anaerobic, thermophilic microorganisms. Other aspects of the present invention are described in detail herein.

### Electroporation

Electroporation is a term describing transport of hydrophilic molecules across a hydrophobic membrane via electrically formed pores (electropores).

While some bacteria can be transformed simply by adding a solution of new DNA to culture media, most bacteria require further manipulation to transport the new DNA across the bacteria's cell wall and membrane into the interior of the bacterial cell. One such technique is electroporation.

DNA generally has a negative charge in aqueous solution as it is an acid and liberates hydrogen ions in solution at physiological pH. Accordingly, DNA tends to move towards a positively charged electrode when an electric field is applied to a solution containing DNA. This phenomenon is known as electrophoresis.

A plasmid carrying a desired DNA segment or gene of interest is prepared in aqueous solution of low ionic strength, and added to bacterial cells suspended in an electroporation buffer. The mixture is typically kept on ice to prevent DNA degradation and to avoid overheating the bacteria during electroporation. Electroporation is performed by exposing the mixture of DNA and suspended bacteria to a high-intensity, brief, electric field. The intense field carries DNA molecules across the hydrophilic cell wall by electrophoresis. The intense field also carries DNA molecules through a temporary electropore in the hydrophobic cell membrane into some, but far from all, of the bacteria.

Typically, electroporation is performed by placing the bacterial suspension and the transforming DNA between electrodes of a chilled electroporation cuvette and applying an electric pulse to the cuvette. A high, DC, or RF modulated voltage pulse is applied to the electrodes for a time typically up to several dozen milliseconds.

The plasmid carrying the desired DNA segment or gene of interest can include sequences homologous to portions of the bacterial chromosome. Homologous DNA sequences allow for the desired DNA segment to be inserted into the bacterial chromosome through recombination events. The plasmid can alternatively include DNA segments that code for integrases, enzymes that aid in the incorporation of portions of the plasmid into the bacterial chromosome. The plasmid can contain DNA genes or elements that allow it to survive and replicate within the bacteria.

The electroporated bacteria are then cultured under conditions that allow them to “recover” from the electroporation event. “Recovery” affords the bacteria time and suitable growth conditions in which they can repair cell structures that may have been damaged during electroporation and to begin expressing genes that will later be used for selection.

The recovered bacteria are then cultured under “selective” conditions favorable to the growth of bacteria that have incorporated the desired new plasmid DNA. Typically, a gene encoding for resistance to an antibiotic is included in the plasmid DNA, and that same antibiotic is included in a post-electroporation culture “selection” media. As a consequence of this selection scheme, transformed bacteria are able to survive and grow in the “selection” media by utilizing and relying on the incorporated antibiotic resistance gene contained within the plasmid DNA molecule while untransformed bacteria remain susceptible to the antibiotic within the selection media.

Alternatively, electroporated bacteria can be cultured into colonies on an agar plate and bacterial products blotted onto a membrane. The membrane can then be stained with fluorescent antibodies to proteins encoded on the desired DNA plasmid. Colonies expressing those proteins will then have associated fluorescent marks on the membrane, thereby allowing identification of colonies that express those proteins.

### Methods of Transforming

A first aspect of the present invention is directed to a method for transforming

DNA into Gram-positive, anaerobic, thermophilic bacteria by electroporation which includes preparing a suspension of bacteria with DNA and applying a voltage burst between a first electrode and a second electrode such that an electric field is applied to the bacterial/DNA suspension such that transformation occurs, wherein the voltage burst comprises one or more identical square pulses that have a duration from about 10 μs to about 3 ms. The DNA that can be transformed into the Gram-positive, anaerobic, thermophilic bacteria is described herein in further detail.

The term “DNA” is intended to encompass a singular deoxyribonucleic acid as well as plural deoxyribonucleic acids, and refers to an isolated nucleic acid molecule or construct, e.g., artificial chromosomes, plasmid DNA, segments of genes. This term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

A DNA molecule can contain the full-length coding nucleotide sequence of a gene, including any endogenous gene promoters, ribosome binding sites or transcription termination sequences. An operable association is when a coding sequence for a gene product, e.g., a polypeptide, is associated with one or more regulatory sequences in such a way as to place expression of the gene product under the influence or control of the regulatory sequence(s).

A “gene” refers to an assembly of nucleotides that encode a polypeptide, and includes cDNA and genomic DNA nucleic acids. “Gene” also refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences.

A DNA “coding sequence” or “coding nucleotide” is a double-stranded DNA sequence which is transcribed and translated into a polypeptide in a cell in vitro or in vivo when placed under the control of appropriate regulatory sequences. Suitable “regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. A promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Inducible promoters are those whose transcriptional activity is induced by the presence or absence of biotic or abiotic factors in the bacterial culture. Inducible promoters include chemically-regulated promoters and physically-regulated promoters. Chemically-regulated promoters include those whose transcriptional activity is regulated by the presence or absence of alcohol, tetracycline, steroids, metal and other compounds. Physically-regulated promoters include those whose transcription activity is regulated by the presence or absence of light and low or high temperatures.

A coding sequence is “under the control” of transcriptional and translational control sequences in a cell when RNA polymerase transcribes the coding sequence into mRNA and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression,” as used herein, refers to the transcription and stable accumulation of sense (mRNA) or antisense RNA derived from the nucleic acid fragment of the invention. Expression may also refer to translation of mRNA into a polypeptide.

A “derivative” of the plasmid of the present invention means a plasmid comprising a part of the plasmid of the present invention, or the plasmid of present invention and another DNA sequence. The “part of a plasmid” means at least a part containing a region essential for autonomous replication of the plasmid. The plasmid of the present invention can replicate in a host microorganism even if a region other than the region essential for the autonomous replication of the plasmid (replication control region), that is, the region other than the region containing the replication origin and genes necessary for the replication, is deleted.

A DNA molecule can comprise a conventional phosophodiester bond or a non-conventional bond (e.g., an amide bond, such as found in peptide nucleic acids (PNA)). The DNA molecule can be a composed of any unmodified or modified deoxyribonucleic acid residues. DNA molecules can also contain one or more modified bases or DNA backbones modified for stability or for other reasons. “Modified” bases include, for example, tritylated bases and unusual bases such as inosine. A variety of modifications can be made to DNA; thus “DNA” embraces chemically, enzymatically, or metabolically modified forms.

Other sequence elements contemplated for use in the transforming DNA are genes which confer survival or metabolic advantages to transformed bacteria so that they can be selected and distinguished from untransformed bacteria. These genes, also referred to as “selectable markers,” create detectable phenotypes which facilitate detection of host cells that contain a plasmid having the selectable marker. Non-limiting examples of selectable markers include drug resistance genes and nutritional markers. For example, the selectable marker can be a gene that confers resistance to an antibiotic selected from the group consisting of: ampicillin, kanamycin, erythromycin, chloramphenicol, gentamycin, kasugamycin, rifampicin, spectinomycin, D-Cycloserine, nalidixic acid, streptomycin, or tetracycline. In one embodiment of the present invention, the expression vector comprises a chloramphenicol acetyltransferase gene. Other non-limiting examples of selection markers include adenosine deaminase, aminoglycoside phosphotransferase, dihydrofolate reductase, hygromycin-B-phosphotransferase, thymidine kinase, and xanthine-guanine phosphoribosyltransferase. A single plasmid can comprise one or more selectable markers.

In certain embodiments, the transforming DNA is contained within an expression vector. As used herein, an “expression vector” and its known variant “expression construct” means a polydeoxyribonucleic acid molecule that is used to introduce and direct the expression a specific gene to which it is operably linked into a target bacterial cell. Expression vectors allow transcription of large amounts of stable mRNA. Once the expression vector is inside the bacterial cell, the Ribo Nucleic Acid (RNA) molecule or protein that is encoded by the gene is produced by the cellular transcription and/or translation machinery. Sequence elements of an expression vector suitable for use in the present invention have been discussed above.

A “shuttle vector” is a cloning vector that is capable of replication and/or expression in more than one host cell type.

In other embodiments of the present invention, the expression vector is a plasmid. As used herein, “plasmid” means an extra-chromosomal element often carrying one or more genes that are not part of the central metabolism of the cell, and is usually in the form of a circular double-stranded DNA molecule. Such elements may be autonomously replicating sequences, genome integrating sequences, phage or nucleotide sequences, linear, circular, or supercoiled, of a single- or double-stranded DNA, derived from any source, in which a number of nucleotide sequences have been joined or recombined into a unique construction which is capable of introducing a promoter fragment and DNA sequence for a selected gene product along with appropriate 3′ untranslated sequence into a cell. Preferably, the plasmids of the present invention are thermostable and self-replicating. Thermostable plasmids suitable for use in the present invention include, for example, those derived from Thermoanaerobacterium saccharolyticum strain B6A.

The terms “heterologous” as used herein refers to an element of a plasmid or cell that is derived from a source other than the endogenous source. Thus, for example, a heterologous sequence could be a sequence that is derived from a different gene or plasmid from the same host, from a different strain of host cell, or from an organism of a different taxonomic group (e.g., different kingdom, phylum, class, order, family genus, or species, or any subgroup within one of these classifications). The term “heterologous” is also used synonymously herein with the term “exogenous.”

In other embodiments, the expression vector comprises a DNA sequence of an endogenous gene of the Gram-positive, anaerobic, thermophilic bacterium to be transformed. Non-limiting examples of endogenous Gram-positive, anaerobic, thermophilic bacterium genes include those found in C. thermocellum. For example, endogenous genes can include those encoding enzymes that function in pyruvate, propionate, butanoate metabolism and variants thereof In other embodiments, the expression vector comprises a DNA sequence of endogenous promoter regions. For example, endogenous gapD and cbp promoter regions can be incorporated into transforming DNA to increase expression of exogenous and endogenous genes of interest. In yet other embodiments, the expression vector comprises a DNA sequence of an exogenous gene to the Gram-positive, anaerobic, thermophilic bacterium to be transformed.

In other embodiments of the present invention, the expression vector to be introduced into the Gram-positive, anaerobic, thermophilic bacterium via electroporation can be extracted from the Gram-positive, anaerobic, thermophilic bacterium.

The term “functional unit” as used herein refers to any sequence which represents a structural or regulatory feature, region, or element. Such functional units, include, but are not limited to a replicon, an origin of replication, a sequence encoding a protein or a functional protein fragment, a restriction site, a multiple cloning site, and any combination thereof. The functional unit may be an untranslated nucleic acid sequence (for example, with regulatory properties or functions) or a sequence for a gene encoding a protein (for example, a structural or regulatory gene).

The term “stable plasmid” refers to a plasmid that is capable of autonomous replication and which is maintained throughout at least one and preferably many successive generations of host cell division. A “thermostable plasmid” is a plasmid that is stable at the temperatures of a thermophilic host.

A “reporter gene” is a gene that produces a detectable product that is connected to a promoter of interest so that detection of the reporter gene product can be used to evaluate promoter function. A reporter gene may also be fused to a gene of interest (e.g., 3′ to the endogenous promoter of the gene of interest), such that the fused genes are expressed as a fusion protein that allow one to detect whether the gene of interest is expressed under a given set of conditions. Non-limiting examples of reporter genes include: β-galactosidase, β-glucuronidase, luciferase, chloramphenicol acetyltransferase (CAT), secreted alkaline phosphatase (SEAP), green fluorescent protein (GFP), red fluorescent protein (RFP), and catechol 2,3-oxygenase (xylE).

The Gram-positive, anaerobic, thermophilic bacteria sought to be transformed by the claimed methods are described herein in further detail. As used herein, bacteria that are defined as “Gram-positive” are those bacteria whose thick cell walls consisting of several layers of peptidoglycan become dehydrated upon treatment with alcohol such that the bacterial cells retain an insoluble crystal violet-iodine complex “Gram” stain despite alcohol extraction.

“Anaerobic” bacteria are those bacteria that lack the appropriate cell machinery to use oxygen as a terminal electron acceptor during respiration. Anaerobic bacteria include aerotolerant anaerobes which can grow in oxygen-rich environments and obligate (or strict) anaerobes which die in the presence of oxygen.

“Thermophilic” bacteria are those bacteria whose growth temperature optimum is above about 45° C. Thermophilic bacteria offer major advantages for biotechnological processes, many of which run more rapidly and efficiently at high temperatures. Higher incubation temperatures increase the diffusion rate and solubilities of non-gaseous compounds of interest and tend to discourage non-thermophilic microbial contamination. Cell culture carried out at high temperatures also eliminates or greatly reduces cooling costs.

In one embodiment of the present invention, the method of transforming Gram-positive, anaerobic, thermophilic bacteria includes transforming bacteria that are Gram-positive, anaerobic, thermophilic bacteria and endospore-forming. In another embodiment, the bacteria are selected from the genus Clostridium, Acinetobacter, Thermoanaerobacterium, and other bacteria having characteristics resembling those of Clostridium species. In yet another embodiment, the bacteria is a Clostridium thermocellum strain. In another embodiment, the bacteria is a Clostridium thermocellum strain selected from the group consisting of DSM 1313, DSM 1237 and DSM 2360.

The method by which the Gram-positive, anaerobic, thermophilic bacteria are electroporated here described herein in further detail. As used herein, the term “transforming,” or variations such as “incorporating,” “introducing,” “transducing,” and “transfecting” means the act of introducing DNA into bacterial cells by a number of techniques known in the art. However, the term “transforming DNA” means a DNA molecule that is to be introduced into bacterial cells. The DNA introduced into the bacterial cell can remain in the cell through several replicative cycles as a transient molecule. The DNA introduced into the bacterial cell can also be integrated into the bacterial cell genome.

As used herein, the term “electroporation,” or variations such as “electrotransformation,” means a method by which bacterial cells are subjected to a brief electrical pulse that causes holes (electropores) to open transiently in their cell walls and membranes such that DNA can enter directly into the bacterial cell cytoplasm.

Electroporation can be performed using apparati known to those of skill in the art. Such apparati have been adequately described, for example, in Tyurin M. V. et al., J. Appl. Microb. 88:220-227 (2000) and International Publication No. WO 2005/116203. Additionally, electroporation apparti and cuvettes suitable for use with the present invention are commercially available through Eppendorf (Electroporator 2510), Bio-Rad (Gene Pulser Xcell System and MicroPulser Electroporator) and Sigma-Aldrich (Electroporator EC100) for example. The commercially available electroporators described here can emit 1-2 square pulses with durations of about 1 ms. One of skill in the art can also modify the electroporator described in International Publication No. WO 2005/116203 to emit more than 2 square pulses with durations of less than 1 ms by outfitting the tetrode switch with any commercially available function generator.

Typically, Gram-positive, anaerobic, thermophilic bacteria are cultured in media at their optimum growth temperature prior to electroporation so that they have entered either the exponential or stationary growth phase of the microbial population growth cycle. The rate of exponential growth is influenced by environmental conditions (temperature, composition of the culture medium) and is a consequence of bacterial cells rapidly dividing. A microbial population has entered the stationary growth phase when either an essential nutrient of the culture medium is used up or some waste product of the organism builds up in the medium to an inhibitory level and exponential growth ceases.

The particular growth phase that a microbial population has entered can be calculated if the generation time is known but is most often measured by optical density. Cell density or optical density measurements are obtained by placing a sample of the bacterial cell culture in a spectrophotometer. With such a device, the cell density or turbidity of the sample is expressed in absorbance units. Exponential and stationary growth phases usually yield microbial populations with cell densities of from about 0.3 to about 1.0. In some embodiments of the present invention, the Gram-positive, anaerobic, thermophilic bacteria to be transformed are cultured prior to electroporation to a cell density OD₆₀₀ of from about 0.3 to about 1.0. In other embodiments, the bacteria are cultured to a cell density OD₆₀₀ of about 0.3, 0.4, 0.5, 0.6, 0.7, 0.8, 0.9, or 1.0. To achieve this cell density, some Gram-positive, anaerobic, thermophilic bacteria have to be cultured from about 12 to about 18 hours. Accordingly, the invention is directed to a method of transforming DNA into Gram-positive, anaerobic, thermophilic bacteria that includes culturing the bacteria prior to electroporation from about 12 to about 18 hours. In one embodiment, the bacteria are cultured prior to electroporation for about 12, 13, 14, 15, 16, 17, or 18 hours.

The Gram-positive, anaerobic, thermophilic bacteria cultured prior to electroporation can be cultured in any media known to those of skill in the art including, but not limited to, M122C, MOPS, SOB, TSY, YMG, YPD, 2XYT, LB, M17, and M9 minimal media.

Once the Gram-positive, anaerobic, thermophilic bacteria have been cultured for the appropriate length of time to the appropriate cell density prior to electroporation, they usually are cooled down to slow or stop cell division. Typically, bacterial cell cultures are cooled to about 4° C. or are placed in eppendorf tubes/cuvettes on ice. The bacterial cell cultures can then be subjected to centrifugation to remove culture media that is not amenable to electroporation. Culture media can contain high concentrations of salts and cell waste products. One consequence of high concentrations of salts in an electroporation buffer is the possibility of the sample “arcing.” Arcing is characterized by a loud “pop” sound and occurs when the sample of DNA and bacteria is extremely conductive of electricity. While arcing can be a function of not washing all of the salt from the culture medium of a bacterial suspension, it can also result in situations where too much DNA is added to the bacterial suspension prior to electroporation, the DNA added to the bacterial suspension prior to electroporation is in a high salt buffer, the bacterial suspension prior to electroporation is too dense with bacteria, the bacterial suspension prior to electroporation contains lysed bacteria, and when electroporation is conducted using cuvettes and/or solutions that have not been sufficiently cooled.

Following the centrifugation of the bacterial cell culture, the centrifuged cells can be washed with electroporation buffer to remove residual salt and suspend the cells in a final solution prior to electroporation. The centrifuged cells can be resuspended in a volume of electroporation buffer that is appropriate for transfer of the entire sample into an electroporation cuvette and would be known to those of skill in the art. For example, the volume can be from about 10 to about 300 μL. While the composition of electroporation buffers are known to those of skill in the art and can be varied to suit the needs of individual bacterial species and electroporation protocols, the electroporation buffer can comprise deionized water autoclaved to remove dissolved oxygen. The electroporation buffer can also comprise, for example, about 50 mM xylose and 5 mM MOPS in reverse-osmosis purified water with a pH of about 7.

Following the resuspension of the centrifuged bacteria in electroporation buffer, a suspension is prepared containing the bacteria and the DNA to be transformed into the bacteria. As used herein, “suspension” means a heterogeneous fluid or sample containing solid particles that are sufficiently large for sedimentation. As this term may be applied to the present invention, a “suspension” comprises DNA and bacterial cells where the bacterial cells are considered sufficiently large for sedimentation. While the person skilled in the art will prepare a suspension with a DNA concentration in mind to minimize arcing and maximize transformation efficiency, the suspension of bacteria and DNA can include from about 1 ng to about 10,000 ng of DNA for example.

While chemicals have been used in the art to weaken the cell walls of bacteria to increase transformation events, the present invention does not rely on such pre-electoporation bacterial cell treatment. Accordingly, the present invention does not rely on bacterial cell treatment with chemicals including, but not limited to, glycine, muralytic enzymes and/or isonicotinic acid hydrazide (isoniacin). In some embodiments of the present invention, the Gram-positive, anaerobic, thermophilic bacteria are not treated with isoniacin prior to electroporation.

The suspension of bacteria and DNA is then transferred to an electroporation cuvette or other such container so that a voltage burst can be applied between a first electrode and a second electrode within the container. While Tyurin describes specially made cuvettes for the transformation of C. thermocellum in International Publication No. WO 2005/116203, cuvettes that are commercially available through Eppendorf, Bio-Rad and Sigma-Aldrich for example, can be used with the present invention. When working with anaerobic bacteria such as C. thermocellum, the cuvette and/or the entire transformation scheme can be kept/performed in an oxygen-free glovebox to minimize exposure of the bacteria to the oxygen-rich ambient environment.

Cuvettes suitable for use in the practice of this invention are any vessels in which electroporation can be performed. Cuvettes of greatest interest are those that fit into automated electroporation apparatus and that contain the electrical connections necessary for passing a current through the cell suspension. Suitable materials of construction are any materials that are electrically insulating, inert to the cell suspension, and able to withstand strong electrical fields and any other conditions that might be encountered in a typical electroporation procedure. Glass, ceramic, and clear plastic such as polycarbonate are examples of suitable materials. Plastic cuvettes are readily formed by molding. Examples of suitable cuvettes are shown in U.S. Pat. No. 5,186,800, in which the electrodes are affixed to the interior surface of, or embedded in, the cuvette walls. The spacing between the electrodes is preferably about 5 mm or less, more preferably from about 1 mm to about 4 mm, and most preferably from about 1.0 mm to about 2.0 mm. The electrodes can be of any configuration, although plate or film electrodes or metal strips are preferred for their ability to produce an electric current over a relatively broad area. Common electrically conductive metals that are corrosion resistant are preferred. Examples are aluminum, silver, gold, and alloys of these metals. The electrode area is preferably from about 5 mm² to about 10 cm², most preferably from about 10 mm² to about 2 cm². The size of the cuvette will preferably be such that the volume between the electrodes, i.e., the volume of the suspension in which electroporation will occur, will range from about 1 μL to about 1 mL, more preferably from about 20 μL to about 500 μL, and most preferably from about 25 μL to about 150 μL.

By applying the voltage burst, an electric field is generated and is applied to the suspension of bacteria and DNA. The resulting electric field causes current to flow through the suspension and into the saline intracellular fluid of the bacteria, burning minute holes, “electropores,” in the bacterial cell walls and electrophoretically transporting DNA molecules in the suspension through the cell walls and into the bacterial cytoplasm. While direct current (DC) voltage bursts that have a duration of greater than 4 ms are commonly used in electroporation methods, alternating current (AC) superimposed on DC voltage bursts have also been successfully described for transforming Clostridium species. (See Klapatch, T. R. et al., J. Indust. Microb. 16:342-347 (1996); Mai V., and Wiegel, J., Appl. Environ. Microb. 66:4817-4821 (2000); Mai, V. et al., FEMS Microb. Lett. 148:163-167 (1997); Peng, H. et al., Biotechnol. Lett. 28:1913-1917 (2006); Tyurin, M. V. et al., Appl. Environ. Microb. 70:883-890 (2004); Tyurin, M. V. et al., Appl. Environ. Microb. 71:8069-8076 (2005); PCT Publication WO 2005/116203; and Tyurin, M. V. et al., J. Appl. Microbial. 88:220-227 (2000).)

In one embodiment of the present invention, the voltage burst is one square pulse that has a duration from about 10 μs to about 3 ms. In another embodiment of the present invention, the voltage burst comprises a series of identical square pulses that have a duration from about 10 μs to about 3 ms. In other embodiments, the voltage burst comprises from about 30 to about 100 identical square pulses having a duration from about 10 μs to about 3 ms. In another embodiment, the voltage burst comprises from about 30 to about 50 identical square pulses having a duration from about 10 μs to about 3 ms.

In one embodiment of the present invention, the square pulse or identical square pulses have field strength from about 15 kV/cm to about 30 kV/cm. In other embodiments, the voltage burst has a duration from about 30 μs to about 1.5 ms. In further embodiments of the present invention, the voltage burst has a duration of about 50 μs, 100 μs, 150 μs, 200 μs, 250 μs, 500 μs, 750 μs, 1 ms, 1.5 ms or 3 ms. In some embodiments of the present invention the voltage burst is a square pulse with an field strength from about 10 kV/cm to about 20 kV/cm. In one embodiment of the present invention, the voltage burst is a square pulse of about 10 kV/cm. In another embodiment, the voltage burst is a square pulse of about 15 kV/cm. In yet another embodiment, the voltage burst is a square pulse of about 20 kV/cm.

One benefit to using short square pulses is that the pulsing scheme results in a more robust transformation protocol as any arc that is generated inside the electroporation cuvette is allowed to extinguish prior to the next voltage pulse applied to the bacterial/DNA suspension. The pulsing scheme disclosed in the present application also minimizes arcing by using voltage bursts with shorter duration times than those observed in the art. Those of skill in the art appreciate the need to minimize arcing during electroporation as the arc, being composed of superheated gas, severely damages bacterial cells in its vicinity.

Another aspect of the present invention is directed to a method for transforming DNA into Gram-positive, anaerobic, thermophilic bacteria by electroporation which includes preparing a suspension of bacteria with DNA and applying a voltage burst between a first electrode and a second electrode such that an electric field is applied to the bacteria/DNA suspension such that transformation occurs, wherein the resulting transformed bacteria are cultured at a recovery temperature followed by culture at a selection temperature, and wherein the recovery temperature is lower than the selection temperature.

While “recovery” affords the bacteria time and suitable growth conditions, including the appropriate culture temperature, in which they can repair cell structures that may have been damaged during electroporation and express genes necessary for subsequence selection, “recovery temperature” means the culture temperature at which cells are cultured just after being subjected to an electric field via electroporation.

In some embodiments of the present invention, the Gram-positive, anaerobic, thermophilic bacteria are cultured after electroporation at a recovery temperature of from about 25° C. to about 52° C. In other embodiments, the bacteria are cultured after electroporation at a recovery temperature of from about 40° C. to about 52° C. In yet another embodiment, the bacteria are cultured after electroporation at a recovery temperature of about 51° C. In another embodiment, the bacteria are cultured after electroporation at a recovery temperature of about 50° C. In some embodiments of the present invention, the Gram-positive, anaerobic, thermophilic bacteria are cultured for at least about 12 hours after electroporation at a recovery temperature. In other embodiments, the bacteria can be cultured for at least about 22 hours after electroporation at a recovery temperature. In other embodiments, the bacteria can be cultured for at least 24 hours after electroporation at a recovery temperature.

The recovering Gram-positive, anaerobic, thermophilic bacteria cultured after electroporation can be cultured in any media known to those of skill in the art including, but not limited to, M122C, MOPS, SOB, TSY, YMG, YPD, 2XYT, LB, M17, and M9 minimal media.

While “selection” refers generally to promoting the growth and propagation of transformed bacteria that are able to survive and grow in the selection media by utilizing and relying on genes contained within the incorporated DNA, the present invention is not limited to schemes that rely on propagating transformed bacteria solely through acquired antibiotic resistance. In such schemes, untransformed bacteria remain susceptible to the antibiotic within the selection media. In one embodiment of the present invention, the claimed method further comprises selecting transformed Gram-positive, anaerobic, thermophilic bacteria by incubating the transformed bacteria on media containing at least one antibiotic for which a transformed bacterium is resistant and for which a non-transformed bacterium is susceptible. As described above, any media known to those of skill in the art can be used for cell cultured during selection including, but not limited to, M122C, MOPS, SOB, TSY, YMG, YPD, 2XYT, LB, M17, and M9 minimal media.

Antibiotics that can be used in selection media include, but are not limited to, ampicillin, chloramphenicol, kanamycin, erythromycin and thiamphenicol. In one embodiment of the present invention, the selection media contains thiamphenicol. In another embodiment of the present invention, the “selection” media contains thiamphenicol at a concentration of about 3 μg/mL of media.

The present invention also considers the use of genes in transforming DNA that confer additional metabolic advantages to the transformed bacteria as compared to the untransformed bacteria. A classic example includes auxotrophic bacteria incapable of synthesizing a particular organic compound required for growth that have been transformed with genes encoding the necessary enzymes allowing the transformed auxotrophic bacteria to survive on minimal nutrient media.

In some embodiments of the present invention, the Gram-positive, anaerobic, thermophilic bacteria are cultured at a selection temperature from about 36 to about 72 hours. In other embodiments, the bacteria are cultured from about 48 to about 72 hours.

As used herein, “selection temperature” means the culture temperature at which cells are cultured just after “recovery.” In the present invention, the recovery temperature is lower than the selection temperature. In some embodiments of the present invention, the Gram-positive, anaerobic, thermophilic bacteria are cultured after electroporation and recovery at a selection temperature of from about 52° C. to about 64° C. In other embodiments, the bacteria are cultured at a selection temperature of about 55° C.

Unlike other electroporation procedures, the present invention includes the use of different recovery and selection temperatures following bacterial cell electroporation. In the present invention, optimal recovery temperature is different and lower than the optimal selection temperature. Not only does the recovery and selection scheme of the present invention result in greater transformation efficiency, it allows electroporated cells to recover for a longer period of time so that any cellular structures damaged as a consequence of electroporation can be repaired and any genes necessary for selection can be expressed. When the electroporated Gram-positive, anaerobic, thermophilic bacteria of the present invention are cultured at recovery temperatures below 52° C. and/or below optimal growth temperatures, the cells can devote more of their cellular resources to cell repair and expression of selection genes which leads to greater cell survival. When electroporated bacterial cells are grown at optimal growth temperatures, intracellular machinery is used not only to repair damaged cellular structures but also to prepare cells for replication. Accordingly, the electroporation methods of the present invention yield more reliable transformation results than those previously described in the art.

The methods of the present invention have also unexpectedly led to increased transformation efficiency in difficult to transform Gram-positive, anaerobic, thermophilic bacteria. In some embodiments of the present invention, the Gram-positive, anaerobic, thermophilic bacteria are transformed with a transformation efficiency of from about 8×10³ CFU/μg DNA to about 5×10⁵ CFU/μg DNA. As used herein, “transformation efficiency” relates to the number of colony forming units (CFU) generated by 1 μg of supercoiled plasmid DNA in a transformation reaction.

Finally, the methods of the present invention are superior to those described in the art as they do not require the use of specially-made or proprietary apparati and can be performed with commercially available apparati.

### Transformed Bacteria

Another embodiment of the present invention is directed to a Gram-positive, anaerobic, thermophilic bacterium transformed with an expression vector by the transformation methods disclosed in the present application.

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

Although methods and materials similar or equivalent to those described herein can be used in practice or testing of the present invention, suitable methods and materials are described below. The materials, methods and examples are illustrative only, and are not intended to be limiting. Other features and advantages of the invention will be apparent from the detailed description and from the claims.

Throughout this specification and claims, the word “comprise,” or variations such as “comprises” or “comprising,” will be understood to imply the inclusion of a stated integer or group of integers but not the exclusion of any other integer or group of integers.

In order to further define this specification, the following additional terms and definitions are herein provided.

It is to be noted that the term “a” or “an” entity, refers to one or more of that entity; for example, “a transformed Gram-positive, anaerobic, thermophilic bacteria,” is understood to represent one or more transformed Gram-positive, anaerobic, thermophilic, bacterial cells. As such, the terms “a” (or “an”), “one or more,” “one or several” and “at least one” can be used interchangeably herein.

As used herein, the term “consists of,” or variations such as “consist of” or “consisting of,” as used throughout the specification and claims, indicate the inclusion of any recited integer or group of integers, but that no additional integer or group of integers can be added the specified method, structure or composition.

### EXAMPLES

### Example 1

In order to establish an initial C. thermocellum bacterial culture, 5 μL of a freezer stock of C. thermocellum DSM 1313 cells were inoculated into 150 mL of M122C medium. These cells were allowed to grow at 55-60° C. until the optical density of the cell culture measured at 600 nm reached a value of 0.3-0.8. To obtain this cell density, cells are usually cultured for about 16 hours.

After the desired optical density was reached, the bacterial cells were placed on ice for 10-15 minutes to halt metabolic activity such as spore formation.

The bacterial cells were centrifuged to remove the M122C media. The pelleted cells were resuspended in 150 mL of a wash buffer (deionized water autoclaved to remove oxygen). The resuspended cells were once again centrifuged to pellet the cells and remove the wash buffer. Again, the pelleted cells were resuspended in 150 mL of wash buffer and subjected to centrifugation to remove any residual salts left over from the M122C culture media.

The pelleted cells were then resuspended in 50-300 μL of wash buffer. 20 μL of the resuspended bacterial cells were transferred into a standard 1 mm electroporation cuvette with 400 ng of plasmid pMU102. The cuvette was then placed in the sample holder of an electroporator comprising a high voltage capacitor and an IGBT switch.

A series of 30-50 square pulses with a duration of 33 μs, a period of 330 μs and a field strength of 19 kV/cm was applied to the electroporation cuvette.

The electroporated suspension was then transferred to 5 mL of M122C media and allowed to recover for 24 hours at a recovery temperature of 51° C.

Transformed C. thermocellum cells were selected after recovery on solid M12CC media with a thiamphenicol concentration of 3 μg/mL and incubated at 55° C. until colonies appeared on the plates (2-3 days). This transformation protocol yielded a transformation efficiency of ˜2×10⁵ CFU/μg DNA.

### Example 2

C. thermocellum M0074 cells were prepared for electroporation as described above in Example 1.

The bacterial cells were centrifuged to remove M122C media and the pelleted cells were resuspended in 150 mL of a wash buffer (deionized water autoclaved to remove oxygen). The resuspended cells were once again centrifuged to pellet the cells and remove the wash buffer. Again, the pelleted cells were resuspended in 150 mL of wash buffer and subjected to centrifugation to remove any residual salts left over from the M122C culture media.

The pelleted cells were then resuspended in 50-300 μL of wash buffer. 20 μL of the resuspended bacterial cells were transferred into a standard 1 mm electroporation cuvette with either no DNA, 40 ng of plasmid pMU102 DNA extracted from C. thermocellum or 400 ng of plasmid pMU102 DNA extracted from E. coli. The cuvette was then placed in the sample holder of an electroporator comprising a high voltage capacitor and an IGBT switch.

A series of 45 square pulses with a duration of 30 μs, and a field strength of 20 kV/cm was applied to the electroporation cuvette.

The electroporated suspension was then transferred to 2 mL of M122C media and allowed to recover for 16 hours at a recovery temperature of 51° C.

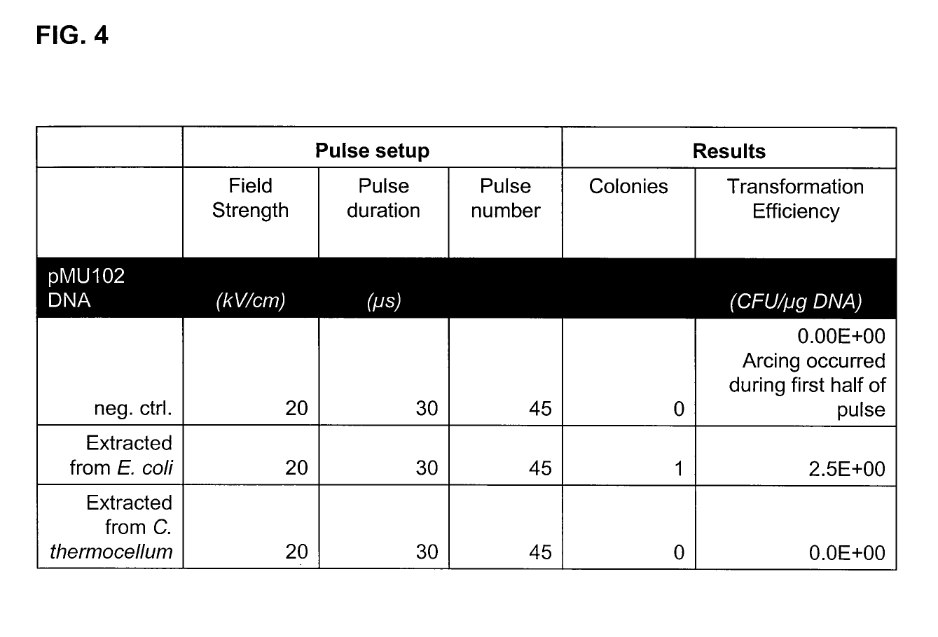

Transformed C. thermocellum M0074 cells were selected after recovery on solid M122C media with a thiamphenicol concentration of 3 μg/mL and incubated at 55° C. for 5 days. FIG. 4 lists the transformation efficiencies observed.

### Example 3

C. thermocellum M0042 cells were prepared for electroporation as described above in Example 1.

The bacterial cells were centrifuged to remove M122C media and the pelleted cells were resuspended in 150 mL of a wash buffer (50 mM xylose, 5 mM MOPS, pH 7). The resuspended cells were once again centrifuged to pellet the cells and remove the wash buffer. Again, the pelleted cells were resuspended in 150 mL of wash buffer and subjected to centrifugation to remove any residual salts left over from the M122C culture media.

The pelleted cells were then resuspended in 50-300 μL of wash buffer. 20 μL of the resuspended bacterial cells were transferred into a standard 1 mm electroporation cuvette with either no DNA or 400 ng of plasmid pMU102. The cuvette was then placed in the sample holder of an electroporator comprising a high voltage capacitor and an IGBT switch.

One square pulse with a duration of 1.5 ms, and a field strength of 15 kV/cm was applied to the electroporation cuvette.

The electroporated suspension was then transferred to 2 mL of M122C media and allowed to recover for 18 hours at a recovery temperature of 50° C.

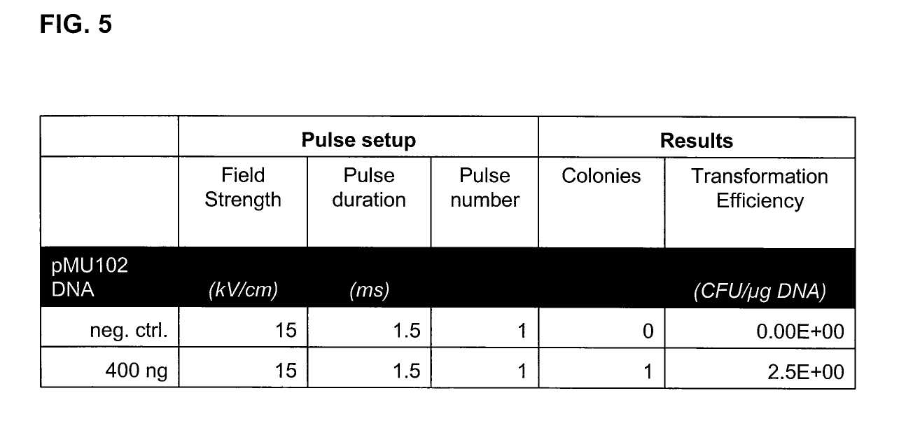

Transformed C. thermocellum M0042 cells were selected after recovery on solid M122C media with a thiamphenicol concentration of 6 μg/mL and incubated at 55° C. for 5 days. FIG. 5 lists the transformation efficiencies observed.

### Example 4

C. thermocellum M0043 cells were prepared for electroporation as described above in Example 1.

The bacterial cells were centrifuged to remove M122C media and the pelleted cells were resuspended in 150 mL of a wash buffer (50 mM xylose, 5 mM MOPS, pH 7). The resuspended cells were once again centrifuged to pellet the cells and remove the wash buffer. Again, the pelleted cells were resuspended in 150 mL of wash buffer and subjected to centrifugation to remove any residual salts left over from the M122C culture media.

The pelleted cells were then resuspended in 50-300 μL of wash buffer. 20 μL of the resuspended bacterial cells were transferred into a standard 1 mm electroporation cuvette with either no DNA or 400 ng of plasmid pMU102. The cuvette was then placed in the sample holder of an electroporator comprising a high voltage capacitor and an IGBT switch.

One square pulse with a duration of 1.5 ms, and a field strength of 15 kV/cm was applied to the electroporation cuvette. Alternatively, 33 square pulses with a duration of 45 μs and a field strength of 22 kV/cm was applied to the electroporation cuvette.

The electroporated suspension was then transferred to 2 mL of M122C media and allowed to recover for ˜12 hours at a recovery temperature of 50° C.

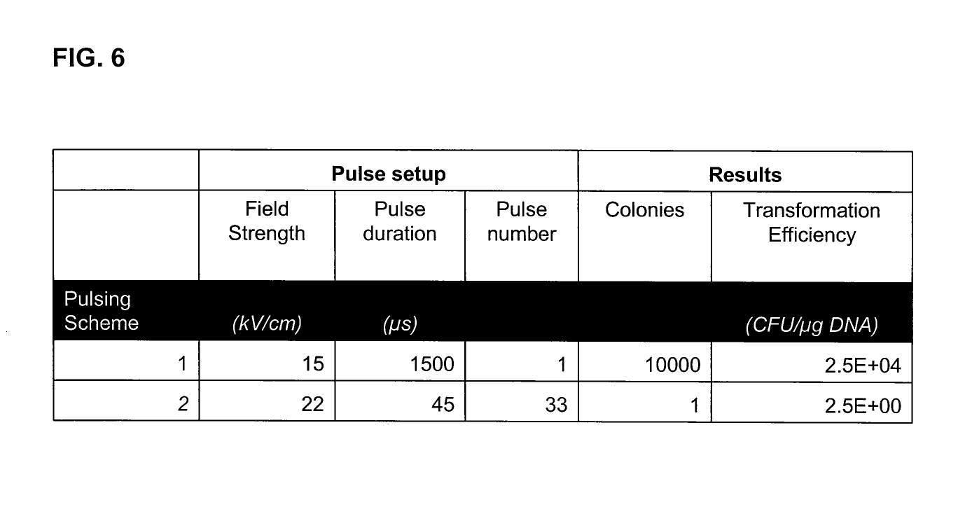

Transformed C. thermocellum M0043 cells were selected after recovery on solid M122C media with a thiamphenicol concentration of 3 μg/mL and incubated at 55° C. until colonies appeared on the plates (2-3 days). FIG. 6 lists the transformation efficiencies observed.

### Example 5

C. thermocellum DSM 1313 cells were prepared for electroporation as described above in Example 1.

The bacterial cells were centrifuged to remove M122C media and the pelleted cells were resuspended in 150 mL of a wash buffer (50 mM xylose, 5 mM MOPS, pH 7). The resuspended cells were once again centrifuged to pellet the cells and remove the wash buffer. Again, the pelleted cells were resuspended in 150 mL of wash buffer and subjected to centrifugation to remove any residual salts left over from the M122C culture media.

The pelleted cells were then resuspended in 50-300 μL of wash buffer. 20 μL of the resuspended bacterial cells were transferred into a standard 1 mm electroporation cuvette with 400 ng of plasmid pMU102. The cuvette was then placed either in the sample holder of an electroporator comprising a high voltage capacitor and an IGBT switch or in the sample holder of a BioRad Gene Pulser Xcell electroporator.

One square pulse with a duration of 1.5 ms, and a field strength of 15 kV/cm was applied to the electroporation cuvette.

The electroporated suspension was then transferred to 2 mL of M122C media and allowed to recover for 24 hours at a recovery temperature of 50° C.

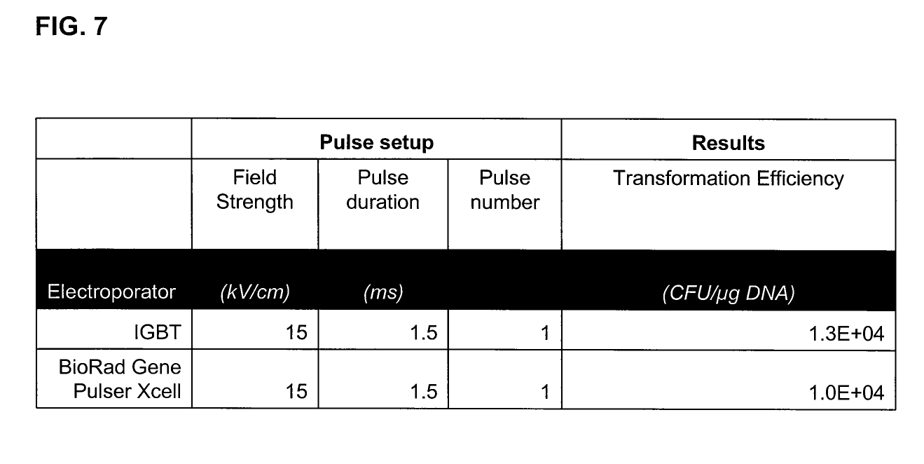

Transformed C. thermocellum DSM 1313 cells were selected after recovery on solid M122C media with a thiamphenicol concentration of 3 μg/mL and incubated at 55° C. until colonies appeared on the plates (2-3 days). FIG. 7 lists the transformation efficiencies observed.

### Example 6

C. thermocellum M0003 cells were prepared for electroporation as described above in Example 1.

The bacterial cells were centrifuged to remove M122C media and the pelleted cells were resuspended in 150 mL of a wash buffer (50 mM xylose, 5 mM MOPS, pH 7). The resuspended cells were once again centrifuged to pellet the cells and remove the wash buffer. Again, the pelleted cells were resuspended in 150 mL of wash buffer and subjected to centrifugation to remove any residual salts left over from the M122C culture media.

The pelleted cells were then resuspended in 50-300 μL of wash buffer. 20 μuL of the resuspended bacterial cells were transferred into a standard 1 mm electroporation cuvette with 0.16, 0.8, 4, 20 or 100 ng of plasmid pNW33N isolated from E. coli. The cuvette was then placed either in the sample holder of an electroporator comprising a high voltage capacitor and an IGBT switch or in the sample holder of a BioRad Gene Pulser Xcell electroporator.

One square pulse with a duration of 1.5 ms, and a field strength of 15 kV/cm was applied to the electroporation cuvette.

The electroporated suspension was then transferred to 1-4 mL of M122C media and allowed to recover for 12-18 hours at a recovery temperature of 51° C.

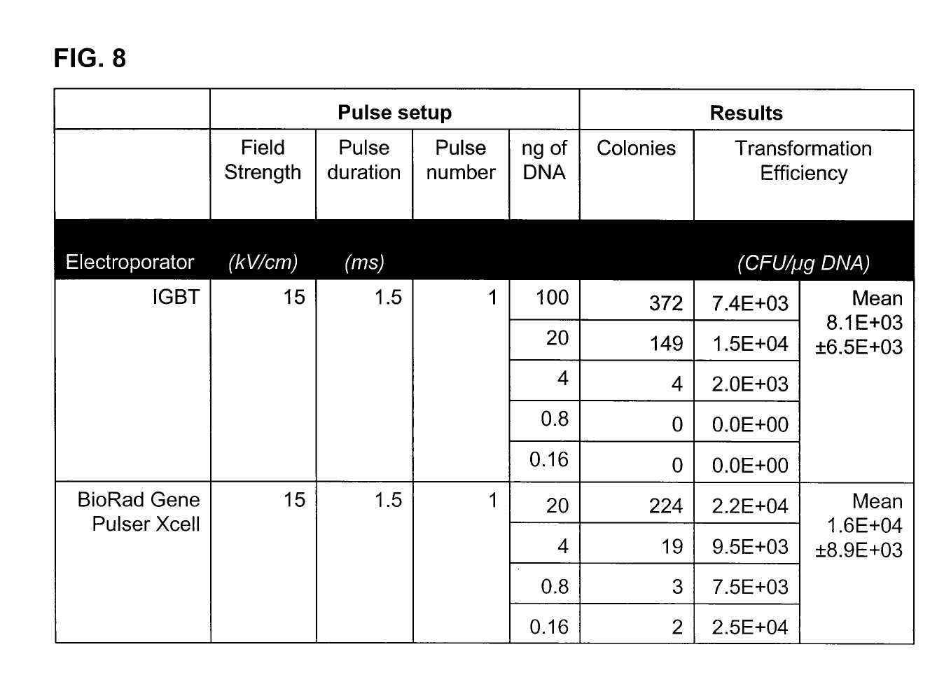

Transformed C. thermocellum M0003 cells were selected after recovery on solid M122C media with a thiamphenicol concentration of 6 μg/mL and incubated at 55° C. until colonies appeared on the plates (2-3 days). FIG. 8 lists the transformation efficiencies observed.

### Example 7

C. thermocellum M0003 cells were prepared for electroporation as described above in Example 1.

The bacterial cells were centrifuged to remove M122C media and the pelleted cells were resuspended in 150 mL of wash buffer 1 (deionized water autoclaved to remove oxygen), wash buffer 2 (50 mM xylose), wash buffer 3 (50 mM xylose, resazurin), wash buffer 4 (50 mM xylose, resazurin, cysteine), wash buffer 5 (500 mM sucrose), or wash buffer 6 (50 mM autoclaved xylose). The resuspended cells were once again centrifuged to pellet the cells and remove the wash buffer. Again, the pelleted cells were resuspended in 150 mL of wash buffer and subjected to centrifugation to remove any residual salts left over from the M122C culture media.

The pelleted cells were then resuspended in 50-300 μL of wash buffer. 20 μL of the resuspended bacterial cells were transferred into a standard 1 mm electroporation cuvette with 50 ng of plasmid pMU102 isolated from C. thermocellum. The cuvette was then placed in the sample holder of an electroporator comprising a high voltage capacitor and an IGBT switch.

45 identical square pulses with a duration of 30 μus, and a field strength of 15 kV/cm was applied to the electroporation cuvette.

The electroporated suspension was then transferred to 2 mL of M122C media and allowed to recover for 12-18 hours at a recovery temperature of 51° C.

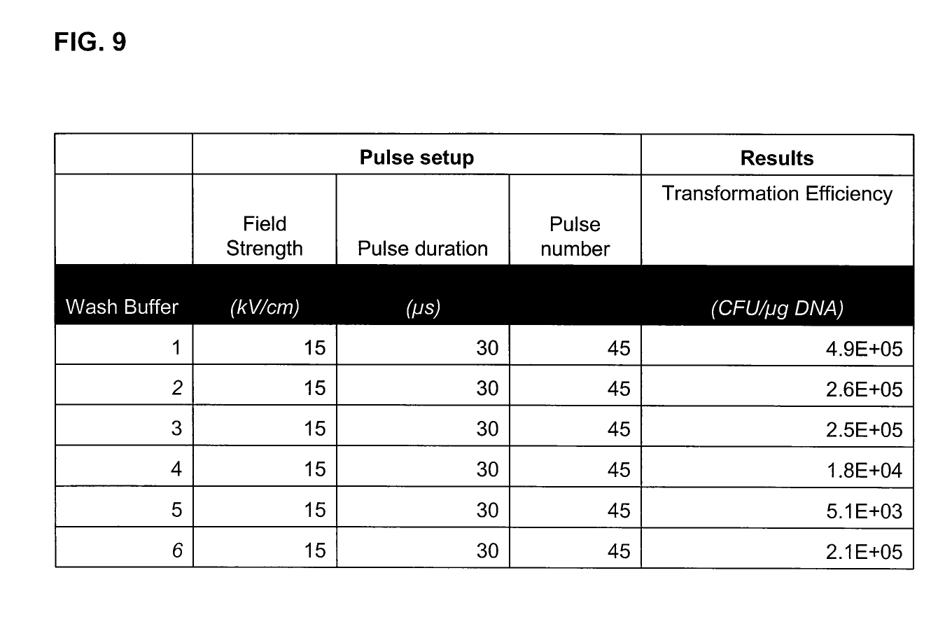

Transformed C. thermocellum M0003 cells were selected after recovery on solid M122C media with a thiamphenicol concentration of 6 μg/mL and incubated at 55° C. until colonies appeared on the plates (2-3 days). FIG. 9 lists the transformation efficiencies observed.

1. A method for transforming DNA into Gram-positive, anaerobic, thermophilic bacteria by electroporation comprising:
preparing a suspension of said bacteria with said DNA; and
applying a voltage burst between a first electrode and a second electrode such that an electric field is applied to said suspension of said bacteria and DNA such that transformation occurs, wherein said voltage burst comprises one or more identical square pulses, and wherein each square pulse has a duration from about 10 μs to about 3 ms.

2. A method for transforming DNA into Gram-positive, anaerobic, thermophilic bacteria by electroporation comprising:
preparing a suspension of said bacteria with said DNA; and
applying a voltage burst between a first electrode and a second electrode such that an electric field is applied to said suspension of said bacteria and DNA such that transformation occurs, wherein said bacteria are cultured at a recovery temperature followed by culture at a selection temperature, and wherein said recovery temperature is lower than said selection temperature.

3. The method according to claim 1, wherein said voltage burst comprises one square pulse.

4. The method according to claim 1, wherein said voltage burst comprises from about 30 to about 100 identical square pulses.

5. The method according to claim 4, wherein said voltage burst comprises from about 30 to about 50 identical square pulses.

6. The method according to claim 1, wherein said square pulse has a field strength from about 15 kV/cm to about 30 kV/cm.

7. The method according to claim 1, wherein said identical square pulses have a field strength from about 15 kV/cm to about 30 kV/cm.

8. The method according to claim 1, wherein said voltage burst is a square pulse with a field strength from about 10 kV/cm to about 20 kV/cm.

9. The method according to claim 1, wherein said voltage burst has a duration from about 30 μs to about 1.5 ms.

10. The method according to claim 1, wherein said bacteria are cultured prior to electroporation to a cell density OD600 of from about 0.3 to about 1.0.

11. The method according to claim 1, wherein said bacteria are cultured prior to electroporation from about 12 to about 18 hours.

12. The method according to claim 2, wherein said bacteria are cultured for at least about 12 hours after electroporation at a recovery temperature.

13. The method according to claim 12, wherein said bacteria are cultured for at least about 22 hours after electroporation at a recovery temperature.

14. The method according to claim 2, wherein said bacteria are cultured after electroporation at a recovery temperature of from about 25° C. to about 52° C.

15. The method according to claim 14, wherein said bacteria are cultured after electroporation at a recovery temperature of from about 40° C. to about 52° C.

16. The method according to claim 15, wherein said bacteria are cultured after electroporation at a recovery temperature of about 51° C.

17. The method according to claim 2, wherein said bacteria are cultured after electroporation and recovery at a selection temperature of from about 52° C. to about 64° C.

18. The method according to claim 17, wherein said bacteria are cultured at a selection temperature of about 55° C.

19. The method according to claim 2, wherein said bacteria are cultured at a selection temperature from about 36 to about 72 hours.

20. The method according to claim 19, wherein said bacteria are cultured from about 48 to about 72 hours.

21. The method according to claim 1, further comprising selecting said transformed bacteria by incubating said bacteria on media containing at least one antibiotic for which a transformed bacterium is resistant and for which a non-transformed bacterium is susceptible.

22. The method according to claim 21, wherein said media contains thiamphenicol.

23. The method according to claim 1, wherein said bacteria are not treated with isoniacin prior to electroporation.

24. The method according to claim 1, wherein said bacteria are endospore-forming.

25. The method according to claim 24, wherein said bacteria are selected from the genus Clostridium or Thermoanaerobacterium.

26. The method according to claim 25, wherein said bacteria is a Clostridium thermocellum strain.

27. The method according to claim 26, wherein said bacteria are selected from the group of Clostridium thermocellum strains consisting of DSM 1313, DSM 1237 and DSM 2360.

28. The method according to claim 1, wherein said transforming DNA is contained in an expression vector.

29. The method according to claim 28, wherein said expression vector is a plasmid.

30. The method according to claim 28, wherein said expression vector comprises a DNA sequence of an endogenous gene of said Gram-positive, anaerobic, thermophilic bacteria.

31. The method according to claim 28, wherein said expression vector comprises a DNA sequence of an exogenous gene to said Gram-positive, anaerobic, thermophilic bacteria.

32. The method according to claim 28, wherein said expression vector comprises a chloramphenicol acetyltransferase gene.

33. The method according claim 1, wherein said bacteria are transformed with a transformation efficiency of from about 8×10³ CFU/μg DNA to about 5×10⁵ CFU/μg DNA.

34. A transformed Gram-positive, anaerobic, thermophilic bacterium transformed by the method of claim 1.
